(19) United States
(12) Patent Application Publication
(10) Pub. No.: US 2018/0325479 A1
(43) Pub. Date: Nov. 15, 2018

(54) METHOD FOR IMAGING AN EXAMINATION REGION HAVING FIRST CONTRAST MEDIUM AND SECOND CONTRAST MEDIUM

(71) Applicant: Siemens Healthcare GmbH, Erlangen (DE)

(72) Inventors: Thomas FLOHR, Uehlfeld (DE); Andre HARTUNG, Erlangen (DE); Bernhard SCHMIDT, Fuerth (DE)

(73) Assignee: Siemens Healthcare GmbH, Erlangen (DE)

(21) Appl. No.: 15/973,637

(22) Filed: May 8, 2018

(30) Foreign Application Priority Data
May 10, 2017 (EP) 17170450.5
Jan. 17, 2018 (EP) 18152125.3

(51) Int. Cl. A61B 6/00; A61B 6/03; A61K 49/04; A61K 9/00

(52) U.S. Cl. CPC A61B 6/481; A61B 6/032; A61B 6/507; A61B 6/482; A61K 49/0423; A61K 9/0019; A61K 49/0419

(57) ABSTRACT

A method is for imaging an examination region of an object to be examined, the examination region including a first contrast medium and a second contrast medium different from the first contrast medium. In an embodiment, the method includes acquisition and generation. In the acquisition, first projection scan data is acquired in an examination region with a first energy range and second projection scan data, different from the first projection scan data, is acquired with a second energy range different from the first energy range. In the generation, a first image is generated based upon the first projection scan data and the second projection scan data and the first image has furthermore only isolated first information of the first contrast medium, only isolated second information of the second contrast medium, or isolated first information of the first contrast medium together with isolated second information of the second contrast medium.

### PRIORITY STATEMENT

The present application hereby claims priority under 35 U.S.C. § 119 to European patent application numbers EP17170450.5 filed May 10, 2017 and EP18152125.3 filed Jan. 17, 2018, the entire contents of each of which are hereby incorporated herein by reference.

### FIELD

At least one embodiment of the invention generally relates to a method and to an image generating device, to a computed tomography system, to a computer program product and/or to a computer-readable medium for distinguishing or separate representation of information of a first contrast medium and a second contrast medium in a first image.

### BACKGROUND

Energy-resolved image data or images can be generated in X-ray imaging, for example in computed tomography. Counting or energy-resolved X-ray detectors or/and a plurality of X-ray spectra can be used for this purpose. Energy-resolved X-ray detectors can provide energy-resolved attenuation data in a plurality of energy ranges from one scan with a single X-ray spectrum or a plurality of X-ray spectra. The energy ranges can depict the energy deposited in the converter material or, for example following a step of convolution of the energy deposition spectrum via a detector response function, the energy of the X-ray photons. The examination volume can be acquired using a plurality of X-ray spectra, for example in conjunction with at least one integrating X-ray detector. For example, at least two X-ray sources detector systems or at least two different X-ray spectra of a single X-ray source can be used.

The X-ray radiation or the photons can be converted in direct conversion X-ray detectors by a suitable converter material into electrical pulses. For example, CdTe, CZT, CdZnTeSe, CdTeSe, CdMnTe, InP, TlBr₂, HgI₂, GaAs or others can be used as the converter material. The electrical pulses are evaluated by an electronic evaluation device, for example an integrated circuit (Application Specific Integrated Circuit, ASIC). In counting X-ray detectors, incident X-ray radiation is measured by counting the electrical pulses, which are triggered by the absorption of X-ray photons in the converter material. As a rule, the level of the electrical pulses is proportional to the energy of the absorbed X-ray photons. As a result, an item of spectral information can be extracted by comparing the level of the electrical pulse with a threshold value.

Computed tomography is an imaging method, which is primarily used for medical diagnostics and for material examination. In computed tomography, a radiation source, for example an X-ray source, and a detector device cooperating therewith rotate about an object to be examined in order to acquire three-dimensional image data. Scan data is acquired within an angular sector during the rotational movement. The projection scan data is a large number of projections, which contain information about the attenuation of the radiation by the examination object from different projection angles. A two-dimensional sectional image or a three-dimensional volume image of the examination object can be calculated from these projections. The projection scan data is also called raw data, or the projection scan data can already be pre-prepared, so for example detector-based differences in the intensity of the attenuation are reduced. Image data can then be reconstructed from this projection scan data, for example via what is known as filtered back projection or by way of an iterative reconstruction method, and an image created thereby.

From document WO 2016/146214 A1 a system for a transarterial chemoembolization of a region of interest, comprising a tumor, is known. The system comprises an injecting device, which is arranged such that it introduces first medicament-eluting microsphere pearls, which contain at least one first medicament and one first contrast medium, into the region of interest and second medicament-eluting microsphere pearls, which contain at least one second medicament and one second contrast medium, into the region of interest. An imaging system is arranged to obtain a first image data set of the region of interest having at least one first X-ray radiation energy and a second image data set of the region of interest having at least one second X-ray radiation energy. A concentration-determining device is arranged to determine a first medicament concentration from the first image data set and a second medicament concentration from the second image data set.

From document US 2015/0221082 A1 a method is known, which comprises the determination of permeability metrics of the vessel tissue of interest on the basis of a first time amplification curve and a second time amplification curve, which correspond to a first contrast material and a second contrast material, which flow through the vessel tissue of interest and generate a signal which indicates this. A computing system comprises a time amplification curve generator, which receives first dynamically contrast-enhanced imaging data, which displays the vessel tissue of interest and a first contrast material having weakly penetrating particles, and the second dynamically contrast-enhanced imaging data, which display the vessel tissue of interest and a second contrast material having strongly penetrating particles. The computing system generates a first time amplification curve for the first contrast material and a second time amplification curve for the second contrast material. A permeability metrics-determining device determines permeability metrics for the vessel tissue of interest in that an effective difference is determined between the first and second time amplification curves.

From document US 2015/0038827 A1 a medical image diagnostic device, an imaging unit, an image generating unit and a display unit are known. The image generating unit images a subject, into which blood vessel contrast-enhancing particles and diseased tissue contrast-enhancing particles were injected. The blood vessel contrast-enhancing particles have the first particle size, which is greater than the gap in the vascular endothelial cells under the EPR effect. The diseased tissue contrast-enhancing particles have the second particle size smaller than the gap. The image generating unit generates a medical image, which is connected to an imaging region of the subject, based on output data of the imaging unit. The display unit displays the medical image.

### SUMMARY

At least one embodiment of the invention addresses a problem that until now no possibility has existed for quantifying contrast medium simultaneously present in the body, for example in connection with nanoparticles or microspheres, in a scan or an image. As a result, a first contrast medium cannot be used for example in preceding interventions since the second contrast medium cannot differ from the first contrast medium in the image.

Embodiments of the invention disclose a method, an image generating device, a computed tomography system, a computer program product and/or a computer-readable medium, which enable a distinction or separate display of information of a first contrast medium and a second contrast medium in a first image.

At least one embodiment of the invention relates to a method for imaging an examination region of an object to be examined, wherein the examination region has a first contrast medium and a second contrast medium different from the first contrast medium, with a computed tomography system, having the steps of acquisition of first projection scan data and second projection scan data and generation of a first image from the first projection scan data and the second projection scan data. Acquisition comprises acquiring the first projection scan data having a first energy range and the second projection scan data different from the first projection scan data having a second energy range different from the first energy range in an examination region. Generation comprises generating the first image on the basis of the first projection scan data and on the basis of the second projection scan data. Generation of the first image supplies a first image having only isolated first information of the first contrast medium, only isolated second information of the second contrast medium, or isolated first information of the first contrast medium together with isolated second information of the second contrast medium. The first image can for example additionally comprise an attenuation image of the examination region. Attenuation by the first contrast medium or/and the second contrast medium can be overlooked or be displayed in the first image by the isolated first information or the isolated second information. The attenuation image can for example be used for anatomical orientation. The first image can for example additionally have a marking of a region of interest, in which treatment is carried out by way of the therapeutically effective functionalization of the first contrast medium. The isolated first item of information or isolated second item of information can be displayed in the first image, for example as gray stages or colored shadings, or as a numerical value.

At least one embodiment of the invention relates, moreover, to an image generating device for carrying out at least one embodiment of the inventive method, having an input interface for acquiring projection scan data from an examination region of an object to be examined, obtained via a computed tomography system with the aid of a scan, a reconstruction unit for reconstructing a first image on the basis of the acquired first projection scan data and second projection scan data, and an image data interface for outputting the first image. All steps of at least one embodiment of the inventive method can advantageously be carried out in the image generating device.

At least one embodiment of the invention relates, moreover, to a computed tomography system, having a projection data acquisition unit, comprising an X-ray source and a detector device for acquiring projection scan data of an examination region of an object to be examined, a controller for controlling the projection data acquisition unit, and at least one embodiment of an inventive image generating device. The computed tomography system advantageously has all componenets for carrying out at least one embodiment of the method.

At least one embodiment of the invention relates, moreover, to a computer program product having a computer program, which can be loaded directly into a storage device of a controller of a computed tomography system, having program segments in order to carry out all steps of at least one embodiment of the inventive method when the computer program is executed in the controller of the computed tomography system. The computer program product can advantageously be used such that a computed tomography system can carry out at least one embodiment of the inventive method.

At least one embodiment of the invention relates, moreover, to a computer-readable medium, on which program segments that can be read and executed by an arithmetic unit are stored in order to carry out all steps of at least one embodiment of the inventive method when the program segments are executed by the arithmetic unit. The computer-readable medium can advantageously be used such that a computed tomography system can carry out at least one embodiment of the inventive method.

### BRIEF DESCRIPTION OF THE DRAWINGS

Example embodiments of the invention will be illustrated below with reference to drawings, in which:

FIG. 1 schematically shows a concept of an inventive method according to a first embodiment;

FIG. 2 schematically shows a concept of an inventive method according to a second embodiment;

FIG. 3 schematically shows a concept of an inventive method according to a third embodiment;

FIG. 4 schematically shows a concept of an inventive method according to a fourth embodiment;

FIG. 5 schematically shows a concept of an inventive computer tomograph according to a first embodiment; and

FIG. 6 schematically shows a concept of an inventive computer tomograph according to a second embodiment.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the invention relates to a method for imaging an examination region of an object to be examined, wherein the examination region has a first contrast medium and a second contrast medium different from the first contrast medium, with a computed tomography system, having the steps of acquisition of first projection scan data and second projection scan data and generation of a first image from the first projection scan data and the second projection scan data. Acquisition comprises acquiring the first projection scan data having a first energy range and the second projection scan data different from the first projection scan data having a second energy range different from the first energy range in an examination region. Generation comprises generating the first image on the basis of the first projection scan data and on the basis of the second projection scan data. Generation of the first image supplies a first image having only isolated first information of the first contrast medium, only isolated second information of the second contrast medium, or isolated first information of the first contrast medium together with isolated second information of the second contrast medium. The first image can for example additionally comprise an attenuation image of the examination region. Attenuation by the first contrast medium or/and the second contrast medium can be overlooked or be displayed in the first image by the isolated first information or the isolated second information. The attenuation image can for example be used for anatomical orientation. The first image can for example additionally have a marking of a region of interest, in which treatment is carried out by way of the therapeutically effective functionalization of the first contrast medium. The isolated first item of information or isolated second item of information can be displayed in the first image, for example as gray stages or colored shadings, or as a numerical value.

The first projection scan data and the second projection scan data are preferably acquired via a computed tomography system (CT). Acquisition can comprise a sequential scan or a spiral scan of the examination region. The first projection scan data and the second projection scan data indicate the attenuation of the X-ray radiation by the object. The first projection scan data and the second projection scan data indicate essentially a measure of the X-rays radiated through the examination region. The first projection scan data and the second projection scan data can be pre-processed, so for example corrections of the detector response can be considered. The first projection scan data and the second projection scan data preferably comprise scans of the same examination region. The scan can in particular comprise acquisition of the first projection scan data and the second projection scan data of the essentially identical examination region. Acquisition of the first projection scan data or of the second projection scan data can additionally comprise a region around the examination region. The first projection scan data and the second projection scan data can preferably be acquired simultaneously. The first projection scan data and the second projection scan data can be acquired at staggered intervals.

The first projection scan data and the second projection scan data can be used for a step of reconstruction. The reconstruction can for example comprise a filtered back projection or/and an iterative reconstruction. The first image can either depict a first item of information or a second item of information, or a first item of information and a second item of information together, and thereby so as to be distinguishable. The first image comprises in particular the complete examination region.

Within the context of CT examinations, a second, for example iodine-containing, contrast medium, can be administered, as a rule intravenously, for the visualization of vessels or displaying the tissue perfusion. Due to its high atomic number, this leads to a good contrast in CT images. The contrast can in particular be increased with respect to surrounding soft tissue, which essentially has no first contrast medium and no second contrast medium. Enriched structures, lesions or vessels can therefore be demarcated from each other better. Contrast medium can likewise be administered within the context of interventional procedures, it being possible for example for only the display of vessels to be in the foreground. The examination region comprises in particular a region of the examination object relevant, for example anatomically or physiologically, to the type of examination or to the subsequent diagnosis or checking of the result of a treatment, based for example on the first image.

Furthermore, other methods or techniques can also lead to a contrast enhancement in CT images. During the course of studies it was possible to demonstrate that, if they are present in sufficient concentration, nanoparticles can be displayed via a CT scan. For example, nanoparticles based on gold can be enriched in plaques of vessels in a concentration so detection by a clinical CT scan is possible. A different region, in which X-ray-sensitive substances are brought into the body in order to achieve a contrast enhancement, can occur in the course of cancer treatments. With some methods or techniques, a therapeutic agent, for example radioactive microspheres or TARE, can be introduced intra-arterially in the course of an intervention directly into the region of the tumor. To better assess the distribution of the therapeutic agent, the concentration and also the continuance over time, this therapeutic agent can be marked in advance with an element with a higher atomic number, for example holmium, iron or others, or a compound having an atom with a higher atomic number. The corresponding distribution of the therapeutic agent can thus be rendered visible in the CT image or first image.

The method can also comprise the steps of controlling injecting devices of the first contrast medium and the second contrast medium, with each control unit using a particularly patient-specific injection protocol. The arithmetic unit of the computed tomography system can supply parameters, for example of a quantity of the first contrast medium or the second contrast medium or the injection instant, to the injection protocols or/and receive them.

Both the first contrast medium as well as the second contrast medium, for example marked microspheres or nanoparticles, lead to increased X-ray absorption and therewith to higher CT values (in Hounsfield units, HU) in the first image or in the CT images. The inventive method makes it possible for the first contrast medium or the second contrast medium to be displayed alone and to be quantified. As soon as methods are combined, however, for example detection of the perfusion of a tumor by intravenously administered, iodine-containing second contrast medium with the simultaneous presence of for example holmium-containing microspheres as the first contrast medium from a preceding intervention, until now there has been no possibility for distinguishing and quantifying the first contrast medium and the second contrast medium, because the individual substances cannot be separated from each other in the CT image solely on the basis of their CT value.

In at least one embodiment, the inventors are proposing a spectral method of computed tomography for distinguishing a second contrast medium, administered for example arterially or intravenously, from a first contrast medium, for example having microspheres or nanoparticles. Depending on the choice of first contrast medium and second contrast medium, data can be acquired using dual-energy techniques in which the X-ray-sensitive material of the microspheres or nanoparticles would behave like “dense” water, or using multi-energy techniques. For example, a plurality of scans can be acquired in the dual-energy mode or by the use of counting detectors, which enable simultaneous acquisition of a plurality of energy ranges. Particularly advantageously the scan can be with counting detectors, it can have perfect matching of a plurality of images, for example the first image, the second image, the third image, anatomical CT image, contrast medium image or nanoparticle image, in particular by simultaneous acquisition of the first projection scan data and second projection scan data. By applying a K-edge method or a general method of multi-material separation to the first projection scan data and the second projection scan data, the first contrast medium can advantageously be separated from the second contrast medium and the first contrast medium and the second contrast medium can advantageously be quantified despite the presence of the first contrast medium and the second contrast medium. Enriched structures, lesions or vessels can be differentiated from each other better by the second contrast medium, in particular also in the presence of a first contrast medium in the examination region. A waiting time for breaking down a previously administered, in particular first, contrast medium can advantageously be avoided.

Acquisition comprises acquiring at least the first projection scan data with a first energy range and the second projection scan data different from the first projection scan data with a second energy range different from the first energy range in an examination region. Further projection scan data can be acquired, in particular with further energy ranges, moreover. The first, second and optionally further energy ranges are at least partially different. For example, at least two different X-ray spectra can be used as at least two different energy ranges. For example, at least two different detector energy ranges can be used as the at least two different energy ranges. At least two projection scan data, in particular at least two projection scan data sets, can be acquired. The number of energy ranges or projection scan data (sets) can in particular lie in the region of 2 to 10, preferably in the region of 2 to 4. Particularly preferably the number of energy ranges or projection scan data (sets) can be 2, 3 or 4. The number of energy ranges or projection scan data (sets) can be for example in the region of 2 to 8 or in the region of 2 to 6. The number of energy ranges or projection scan data (sets) can in particular be integral. Advantageously, use of at least two energy ranges can increase the spectral resolution of the scan. Particularly advantageously the spectral resolution of the scan can be increased by the use of more than two energy ranges.

In at least one embodiment, the inventors are proposing in particular to separate a contrast substance, bound to microspheres or nanoparticles and administered during an intervention, as a first contrast medium from a second contrast medium, administered intraarterially or intravenously during or after the intervention, by acquiring CT data sets with at least two different X-ray energies.

In at least one embodiment, the inventors have identified that the first contrast medium and the second contrast medium cannot be separated in a single-energy CT examination. By way of spectral computed tomography for example two images can be calculated, namely a first image, which contains only the first contrast medium and which can be used for determining the scope of the treatment, and a second image, which contains only the second contrast medium, and which, because it is a measure of the local blood circulation, can be used for determining the efficacy of the treatment. The spectral computed tomography can comprise a dual-energy approach having two different X-ray spectra or a spectrally resolving or (photon-)counting X-ray detector.

A separation and a quantification of a for example radioactive, deposited therapeutic agent, for example in the course of an intervention (TARE) in the form of microspheres, and a second contrast medium intravenously administered for example during this or following the intervention can advantageously be obtained based on spectral information. The separations and the quantification of nanoparticles, administered for example during the course of an intervention as the first contrast medium, and intravenously administered second contrast medium can be obtained based on spectral information. Depiction of the isolated first information and the isolated second information advantageously cannot be affected by movement between CT examinations.

According to one embodiment of the invention, the first contrast medium has a therapeutically effective functionalization. A therapeutically effective substance can be coupled to the first contrast medium. The first contrast medium can have a therapeutically effective functionalization. The first contrast medium can be in the form of nanoparticles, microspheres or similar and be therapeutically effective either chemically or by radiation. The first contrast medium can be introduced during the intervention, for example into the liver, and remains there. The first contrast medium can advantageously simultaneously have a therapeutically effective functionalization and a contrast medium substance.

According to one embodiment of the invention, the second contrast medium is indicative of a blood flow property. The second contrast medium can indicate a blood flow property in a region or volume of the examination region. The second contrast medium can preferably be a known contrast medium, for example having iodine, gadolinium or others, with which the blood circulation can be examined or displayed. The second contrast medium can be given at the same time or in a follow-up examination after the intervention. The treatment success of the therapeutically effective functionalization can advantageously be displayed or identified. A global blood flow property can also be displayed.

According to one embodiment of the invention, the first image has an item of local information about a local distribution of the first contrast medium or/and second contrast medium. The distribution in the first image can indicate whether or not the first contrast medium or/and the second contrast medium is/are present in local volumes of the examination region. By acquiring the first projection scan data and the second projection scan data, which can also be called CT data sets with at least two different X-ray energies, the local distribution of the first contrast medium administered for example during an intervention and bound to microspheres or nanoparticles, can be displayed and quantified. Furthermore, the local distribution of the second contrast medium administered intraarterially or intravenously for example during or after the intervention can be displayed and quantified. The first contrast medium and the second contrast medium as well as their quantity or local distribution can advantageously be displayed separately from each other. The local distribution or the local information can be displayed in the first image, in the second image or in the third image for example by way of superimposition over an anatomical depiction. The local information or the local distribution can be displayed for example in color or/and by a texture or shading. A global parameter about the local distribution, for example as a sum of the local information, can also be displayed.

According to one embodiment of the invention, the first image has an item of local information about a quantity of the first contrast medium or/and the second contrast medium. The quantity can be an absolute quantity, a density or a concentration. In addition to the distribution, an item of local information about the quantity can also be displayed. The local information can for example be displayed for the entire examination region, a section of the examination region through to a voxel of the first image. A measure of the deposition of the first contrast medium, in particular having therapeutically effective functionalization, in the examination region or treatment area can advantageously be determined and displayed. A measure of the deposition of the second contrast medium, in particular in order to display the blood circulation, in the examination region or treatment area can advantageously be determined and displayed. A global parameter about the quantity, for example as a sum of the local information, can also be displayed.

According to one embodiment of the invention, the first image has an item of local information about a quantity or distribution of the first contrast medium having a therapeutically effective functionalization. The distribution and local density or the local information of the first contrast medium can be used as a measure of the distribution and local quantity or density of chemical or radioactive substances administered during the intervention and bound to the microspheres or nanoparticles, for example Transarterial Chemoembolization (TACE) or Transarterial Radioembolization (TARE), and thereby advantageously as a measure of the scope of the intervention. The first item of information or the distribution and the local density of the second contrast medium administered intraarterially or intravenously during or after the intervention can advantageously be used as a measure of the local blood circulation and thereby of the therapeutic effect. A global parameter about the quantity, for example as a sum of the local information, can also be displayed.

According to one embodiment of the invention, the therapeutically effective functionalization comprises chemical or/and radioactive substances bound to the first contrast medium, or one chemical or/and radioactive substance. The first contrast medium can have chemical substances, for example for Transarterial Chemoembolization (TACE). The first contrast medium can have radioactive substances, for example for Transarterial Radioembolization (TARE). The first contrast medium can advantageously have a plurality of functions such as a therapeutically effective function and contrast enhancement.

Radioembolization or selective internal radio therapy (SIRT) can be used for example for primary and secondary liver tumors. Radioembolization takes place by way of injection of the first contrast medium via for example the hepatic artery. The first contrast medium can have a radioactive substance, for example Yttrium-90. A particularly high concentration or density of the first contrast medium can advantageously be achieved by way of a blood circulation which, as a rule, is stronger in the tumor. The tumor tissue can be killed off by the radioactive substance, so the tumor can at least partially shrink.

According to one embodiment of the invention, the first image has an item of local information about a local distribution of the first contrast medium or/and the second contrast medium in a predetermined region. The predetermined region can be a treatment area. The predetermined region can designate the volume in the examination region that is to be treated with the first contrast medium or its therapeutically effective functionalization. The predetermined region can be marked in the first image, for example by a border or planar marking. The reach of the first contrast medium or success of the introduction in the predetermined region can advantageously be determined by visual comparison or automatic comparison of the predetermined region with local information about a local distribution.

According to one embodiment of the invention, the first image has an item of local information based on the second contrast medium about a local blood circulation in a predetermined region. The local blood circulation in the predetermined region can supply information about the therapeutic effect. The therapeutic effect in the predetermined region can advantageously be determined by visual comparison or automatic comparison of the predetermined region with the local information based on the second contrast medium about the local blood circulation. For example, a tumor can be treated as a predetermined region by the therapeutically effective functionalization. If the predetermined region has a low blood circulation or no blood circulation, then a therapeutic effect can be inferred. Assessment of the therapeutic effect can also comprise biomarkers or a texture analysis, for example of the distribution of the first contrast medium or the second contrast medium.

According to one embodiment of the invention, a second image is generated with isolated second information of the second contrast medium and removed isolated first information of the first contrast medium. A second image can be generated in addition to the first image. Alternatively, the second image can match the first image. The second image can only have isolated second information of the second contrast medium. The first image or the second image can be generated by way of spectral techniques in such a way that the first contrast medium is removed by spectral techniques and only the second contrast medium is displayed. Removal of the first contrast medium can be achieved for example by way of (multi-) material analysis. The blood circulation can advantageously be checked via the second contrast medium in a similar quality to an examination in the absence of the first contrast medium.

According to one embodiment of the invention, a second image is generated having isolated first information of the first contrast medium and removed isolated second information of the second contrast medium. The second image can only have isolated first information of the first contrast medium. The first image or the second image can be generated by way of spectral techniques in such a way that the second contrast medium is removed by spectral techniques and only the first contrast medium is displayed. Removal of the second contrast medium can be achieved for example by way of (multi-) material analysis. The reach of the first contrast medium can advantageously be checked in a similar quality to an examination in the absence of the second contrast medium.

According to one embodiment of the invention, a third image is generated having removed isolated first information of the first contrast medium and having removed isolated second information of the second contrast medium. The first information of the first contrast medium and the second information of the second contrast medium can be removed from the third image by spectral techniques. Only anatomical information without the effect of contrast media can advantageously be displayed in the third image. A more accurate diagnosis, for example in respect of the anatomical surroundings around a region marked with the first contrast medium or the second contrast medium, can advantageously be made in the predetermined region.

According to one embodiment of the invention, the first contrast medium has a contrast substance bound to microspheres or nanoparticles. The microspheres or nanoparticles can advantageously be used as a carrier for the contrast substance, so, together with the therapeutically effective functionalization, it forms the first contrast medium.

According to one embodiment of the invention, the first contrast medium has at least one of the elements holmium, iron, gold, tungsten or gadolinium. The contrast substance bound to the first contrast medium can contain holmium or iron or gold or tungsten or gadolinium. The first contrast medium can have as a contrast substance in particular an element having an atomic number greater than 20. The first contrast medium can have as a contrast substance in particular an element having an atomic number less than 80. The contrast substance satisfies the condition that it is not harmful to the examination object in the dosage required for the examination or treatment. The first contrast medium can advantageously differ from bones.

According to one embodiment of the invention, the second contrast medium has at least one of the elements iodine or gadolinium.

Iodine or gadolinium can be used as the second contrast medium. If the first contrast medium has iodine, the second contrast medium has for example gadolinium. If the first contrast medium has gadolinium, the second contrast medium has for example iodine. The second contrast medium can advantageously be a known, contrast medium suitable for examination of the blood circulation, which is not toxic for example in the required dosage. The first contrast medium and the second contrast medium can advantageously be distinguished by their dissimilarity by way of a spectral technique. The dissimilarity of the two contrast media can be formed for example by different effective cross-sections or attenuation coefficients in respect of the photo or Compton effect. The dissimilarity of the two contrast media can be formed for example in respect of the increased absorption caused by a K-edge of the first contrast medium or the second contrast medium. The two contrast media can be formed differently in such a way that they can be distinguished by way of methods of spectral computed tomography. The two contrast media can in particular have different energy-dependent attenuation coefficients.

According to one embodiment of the invention, the first contrast medium or the second contrast medium has an element, which has a K-edge in the range of 30 to 100 keV. For example, iodine, gadolinium and gold have a K-edge. Furthermore, iodine, gadolinium and gold are suitable as contrast media. The attainable contrast in the for example first image or second image can advantageously be increased by the K-edge of the first contrast medium or the second contrast medium by way of spectral technique, in particular a counting or direct conversion X-ray detector. Either the first contrast medium or the second contrast medium preferably has a K-edge in the range of 30 to 100 keV.

The first energy range or the second energy range can preferably be adjusted to the photon energy, at which the energy value of the K-edge is reached or exceeded. First of all, a first intermediate image can be generated for the first energy range and a second intermediate image for the second energy range. A differential image can then be generated from the first intermediate image and the second intermediate image. The different dependency of the attenuation coefficients on the photon energy of the X-ray radiation of the first contrast medium and the second contrast medium can advantageously be used, with the dependency being particularly pronounced in the region of the K-edge. The choice of the first energy range and the second energy range is particularly advantageous if one of the two energy ranges respectively is above or below the energy value of the K-edge. The differential image can be the first image or the second image. The differential image can only have isolated information of the contrast medium which has an element with a K-edge.

According to one embodiment of the invention, the first energy range comprises a first X-ray spectrum and the second energy range a second X-ray spectrum. The first energy range and the second energy range can be adjusted via two different tube voltages or filters. For example, a dual-energy-capable or/and dual-source computed tomography system can be used. For example, kV switching can be used. The first projection scan data and the second projection scan data can be acquired at staggered intervals or/and spatially offset, for example caused by the changeover in tube voltage or filter or the arrangement of the two X-ray tube detector systems. Two energy ranges that are different from each other can advantageously be provided for the acquisition using simple means, for example the changeover in tube voltage or filter. The first X-ray spectrum can have for example a tube voltage between 70 kV and 100 kV. The second X-ray spectrum can have for example a tube voltage between 120 kV and 150 kV. The first energy range and the second energy range can be designed to partially overlap, in particular in an energy subarea below the maximum photon energy of the first X-ray spectrum, with the tube voltage of the second X-ray spectrum being higher than the tube voltage of the first X-ray spectrum.

According to one embodiment of the invention, the first energy range comprises a first detector energy range and the second energy range a second detector energy range. The first detector energy range and the second detector energy range can preferably be formed in a direct conversion or counting X-ray detector. The first detector energy range or the second detector energy range can be identified by predetermined threshold values. For example, the first detector energy range or the second detector energy range can be identified by one-sided discrimination by way of one threshold value or by two-sided discrimination, what is known as window discrimination, by way of two different threshold values. The first detector energy range and the second detector energy range have registers that are separate from each other for counting the events above the threshold value or between the two threshold values.

In the case of a one-sided discrimination, the first detector energy range and the second detector energy range can at least partially overlap. In the case of a two-sided discrimination, the first detector energy range and the second detector energy range can be essentially separate from each other or be essentially non-overlapping.

The essentially separate detector energy ranges can be separated from each other in the framework of accuracy of discrimination of the electrical signals. The separation is based in particular on the distinction between different electrical signals based on the different deposited energy in the converter material. In respect of the separation of photon energies for example, due to the detector response function, the essential separation of the detector energy ranges can be more inaccurate with respect to separation purely based on the different electrical signals. The first projection scan data and the second projection scan data can advantageously be acquired simultaneously. The X-ray detector can preferably have 2, 3 or 4 detector energy ranges, in particular per detector element or (sub-)pixel. Alternatively or additionally, for example adjacent detector elements or adjacent (sub-)pixels can have different detector energy ranges, with each detector element or each (sub-)pixel having at least one detector energy range.

According to one embodiment of the invention, at least one embodiment of the inventive method comprises a step of multi-material analysis. Multi-material analysis can occur on the basis of raw data or image data. (Multi-)material analysis can utilize for example the different effective cross-sections of different elements for the photo and Compton effect. Two base materials can be chosen. For example, a segmentation-based material quantification with a plurality of material analyses can be performed, it being possible for the first projection scan data and the second projection scan data to be segmented by way of spectral information and for prior knowledge and heuristics to be incorporated.

The first projection scan data and the second projection scan data can be divided into fatty tissue, tissue, dense tissue, bone and air. The first projection scan data and the second projection scan data can preferably be divided into two of fatty tissue, tissue, dense tissue, bone and air and into first contrast medium and second contrast medium.

The first contrast medium and the second contrast medium can advantageously be distinguished from two of fatty tissue, tissue, dense tissue, bone and air. Furthermore, the first contrast medium can be distinguished from the second contrast medium.

Based on the first projection scan data and the second projection scan data, the first contrast medium and the second contrast medium can be distinguished from two of fatty tissue, tissue, dense tissue, bone and air. Identification of bone structures having a low density can advantageously be improved, since some algorithms can distinguish these to only a very limited extent from blood vessels with contrast medium. The volume fractions of the base materials for each region can be output separately by automatic segmenting. A method of multi-material analysis is known for example from document DE102009017615 A1, the entire contents of which are hereby incorporated herein by reference.

More than two materials or pairs of material, for example water or soft tissue, the first contrast medium and the second contrast medium, can advantageously be separated from each other by way of multi-material analysis. For example, water, different tissue types, bone, first contrast medium or second contrast medium can be chosen as the base materials.

At least one embodiment of the invention relates, moreover, to an image generating device for carrying out at least one embodiment of the inventive method, having an input interface for acquiring projection scan data from an examination region of an object to be examined, obtained via a computed tomography system with the aid of a scan, a reconstruction unit for reconstructing a first image on the basis of the acquired first projection scan data and second projection scan data, and an image data interface for outputting the first image. All steps of at least one embodiment of the inventive method can advantageously be carried out in the image generating device.

At least one embodiment of the invention relates, moreover, to a computed tomography system, having a projection data acquisition unit, comprising an X-ray source and a detector device for acquiring projection scan data of an examination region of an object to be examined, a controller for controlling the projection data acquisition unit, and at least one embodiment of an inventive image generating device. The computed tomography system advantageously has all componenets for carrying out at least one embodiment of the method.

At least one embodiment of the invention relates, moreover, to a computer program product having a computer program, which can be loaded directly into a storage device of a controller of a computed tomography system, having program segments in order to carry out all steps of at least one embodiment of the inventive method when the computer program is executed in the controller of the computed tomography system. The computer program product can advantageously be used such that a computed tomography system can carry out at least one embodiment of the inventive method.

At least one embodiment of the invention relates, moreover, to a computer-readable medium, on which program segments that can be read and executed by an arithmetic unit are stored in order to carry out all steps of at least one embodiment of the inventive method when the program segments are executed by the arithmetic unit. The computer-readable medium can advantageously be used such that a computed tomography system can carry out at least one embodiment of the inventive method.

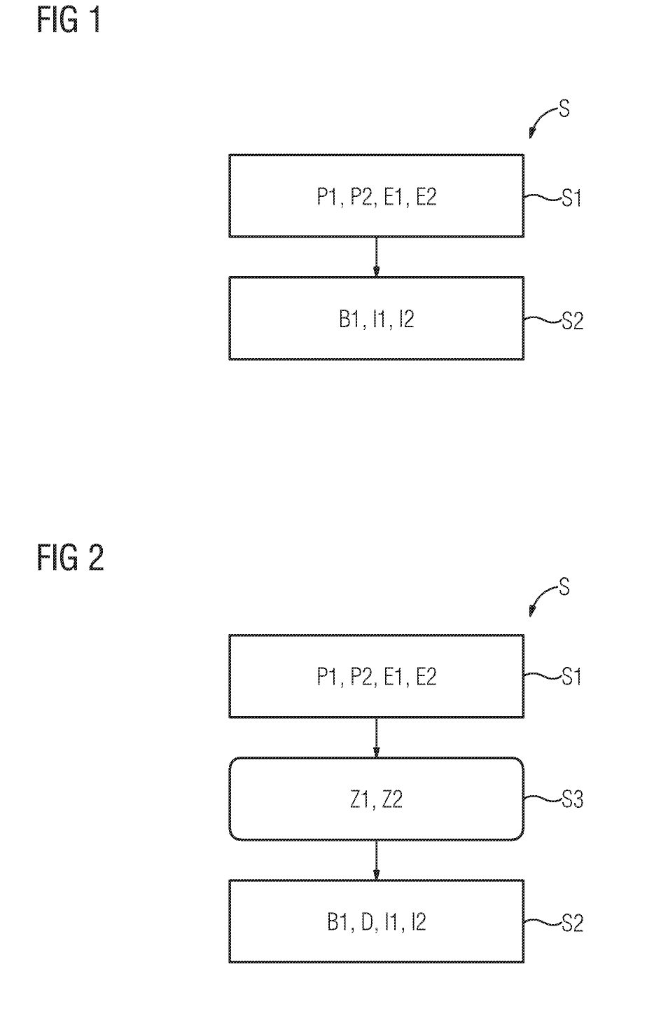

FIG. 1 shows an example implementation of an embodiment of the inventive method S for imaging an examination region of an object to be examined with a computed tomography system according to a first embodiment. The examination region has a first contrast medium and a second contrast medium that is different from the first contrast medium. The method S has at least the steps of acquisition S1 and generation S2. In the step of acquisition S1, first projection scan data P1 is acquired in an examination region with a first energy range E1 and second projection scan data P2 different from the first projection scan data P1 is acquired with a second energy range E2 different from the first energy range E1. The first energy range E1 comprises a first X-ray spectrum and the second energy range E2 comprises a second X-ray spectrum. Additionally or alternatively, the first energy range E1 comprises a first detector energy range and the second energy range E2 a second detector energy range.

Spectral projection scan data P1, P2 is acquired, so a spectral technique for generation S2 of the first image B1 can be applied. In the step of generation S2, a first image B1 is generated on the basis of the first projection scan data P1 and the second projection scan data P2. The first image B1 has, moreover, only isolated first information I1 of the first contrast medium, only isolated second information I2 of the second contrast medium, or isolated first information I1 of the first contrast medium together with isolated second information I2 of the second contrast medium.

The first contrast medium has a therapeutically effective functionalization. The therapeutically effective functionalization comprises chemical or/and radioactive substances bound to the first contrast medium. The first contrast medium has a contrast substance bound to microspheres or nanoparticles. The first contrast medium has at least one, preferably exactly one, of the elements holmium, iron, gold, tungsten or gadolinium. The second contrast medium is indicative of a blood flow property, for example blood circulation. The second contrast medium has at least one, preferably exactly one, of the elements iodine or gadolinium.

The first image B1 can have an item of local information about a local distribution of the first contrast medium or/and second contrast medium. The first image B1 can have an item of local information about a quantity of the first contrast medium or/and of the second contrast medium. The first image B1 can have an item of local information about a quantity or distribution of the first contrast medium having a therapeutically effective functionalization. The first image B1 can have an item of local information about a local distribution of the first contrast medium or/and of the second contrast medium in a predetermined region. The first image B1 can have an item of local information based on the second contrast medium about a local blood circulation in a predetermined region.

FIG. 2 shows an example implementation of an embodiment of the inventive method S for imaging an examination region of an object to be examined with a computed tomography system according to a second embodiment. The method has, moreover, a step of differentiation S3. The first contrast medium or the second contrast medium has an element, which has a K-edge in the range of 30 to 100 keV. For example, iodine, gadolinium and gold have a K-edge in this range. Furthermore, iodine, gadolinium and gold are suitable as contrast media. The attainable contrast in the, for example, first image or second image can advantageously be increased by the K-edge of the first contrast medium or the second contrast medium by way of spectral technique, in particular a counting or direct conversion X-ray detector.

The first energy range E1 or second energy range E2 can preferably be adjusted to the photon energy, at which the energy value of the K-edge is reached or exceeded. First of all, in the step of intermediate image generation S3 a first intermediate image Z1 can be generated for the first energy range E1 and a second intermediate image Z2 can be generated for the second energy range E2. A differential image D can then be generated from the first intermediate image Z1 and the second intermediate image Z2. The different dependency of the attenuation coefficients of the photon energy of the X-ray radiation of the first contrast medium and of the second contrast medium can advantageously be used, with the dependency being particularly pronounced in the region of the K-edge. The choice of the first energy range E1 and the second energy range E2 is particularly advantageous if one of the two energy ranges respectively is above or below the energy value of the K-edge.

With a counting X-ray detector, an energy threshold or discriminator threshold between the first detector energy range and the second detector energy range can be adjusted to the K-edge of the first contrast medium or the second contrast medium. For example, the energy threshold can essentially match the energy of the K-edge. The differential image D can be the first image B1 or the second image B2. The differential image D can only have isolated information of the contrast medium which has an element with a K-edge.

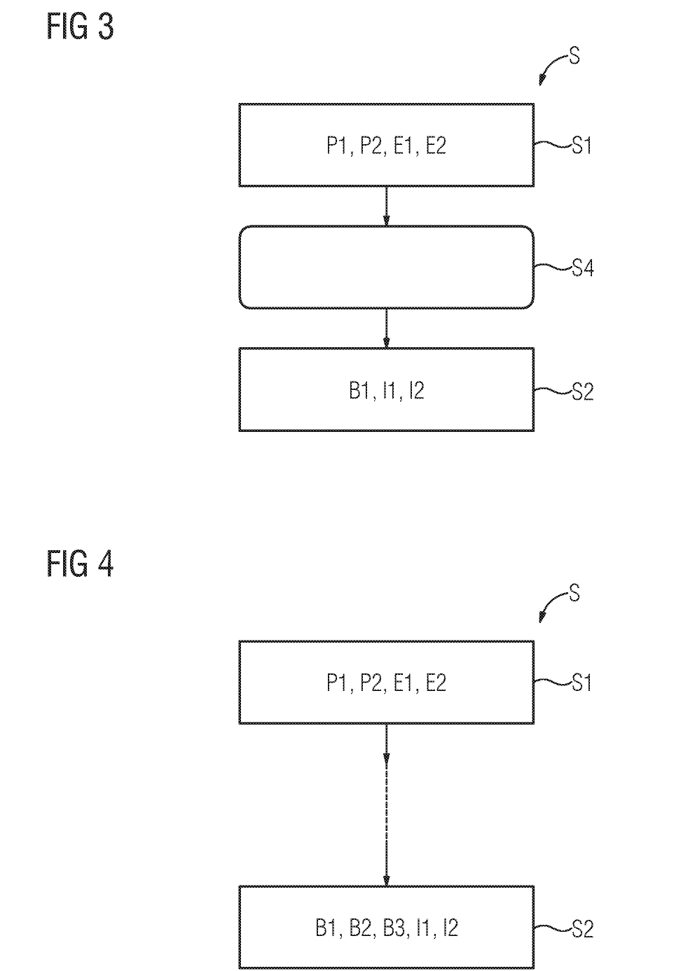

FIG. 3 shows an example implementation of an embodiment of the inventive method S for imaging an examination region of an object to be examined with a computed tomography system according to a third embodiment. The method S also has a step of multi-material analysis S4. Multi-material analysis S4 can take place on the basis of raw data or image data. A method of multi-material analysis S4 is known for example from document DE102009017615 A1. Advantageously more than two materials or pairs of materials can be separated from each other, for example water or soft tissue, the first contrast medium and the second contrast medium.

FIG. 4 shows an example implementation of an embodiment of the inventive method S for imaging an examination region of an object to be examined with a computed tomography system according to a fourth embodiment. The method S according to the first, second and third embodiments can, moreover, generate the second image B2 or/and the third image B3 in the step of generation S2.

The second image B2 has isolated second information I2 of the second contrast medium, wherein the isolated first information I1 of the first contrast medium is removed. Alternatively, the second image B2 has isolated first information I1 of the first contrast medium, with the isolated second information I2 of the second contrast medium being removed. In the third image B3, the isolated first information I1 of the first contrast medium and the isolated second information I2 of the second contrast medium is removed.

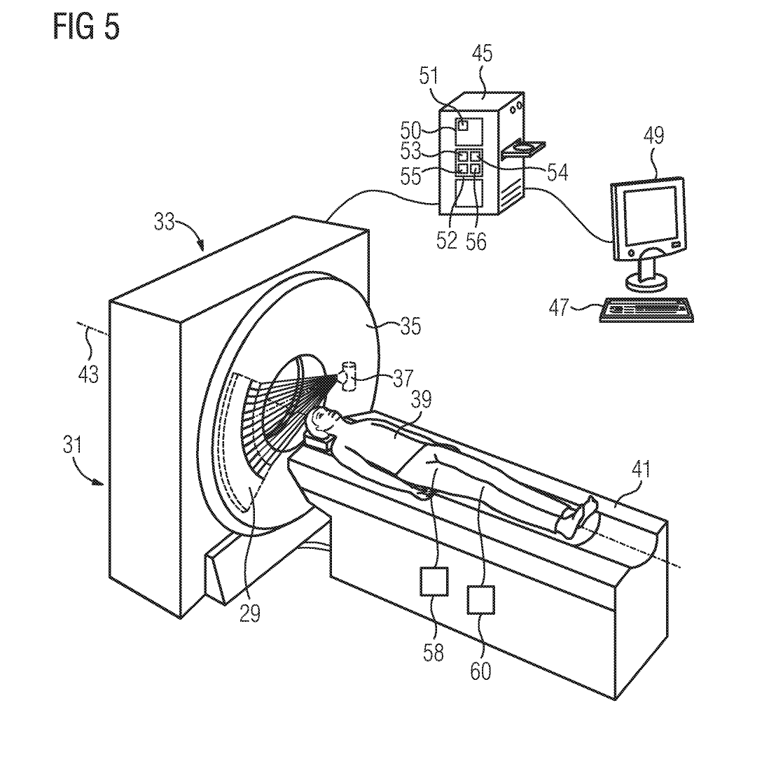

FIG. 5 shows an example implementation of an embodiment of the inventive computed tomography system 31 according to a first embodiment for carrying out embodiments of the inventive method. The computed tomography system 31 includes a projection data acquisition unit 33 having a rotor 35. The rotor 35 comprises an X-ray source 37 and the detector device 29. The object 39 is positioned on the examination table 41 and can be moved along the axis of rotation z 43 through the projection data acquisition unit 33.

An arithmetic unit 45 is used for controlling and calculating the sectional images. The arithmetic unit 45 comprises a controller 50 having a storage device 51. The arithmetic unit 45 comprises, moreover, an image generating device 52 having an input interface 53, a reconstruction unit 54, an image data interface 55 and a material analysis unit 56. The material analysis unit 56 can be suitable for carrying out multi-material analysis or/and the differentiation. An input device 47 and an output device 49 are connected to the arithmetic unit 45.

The computed tomography system 31 also has a first injection device 58 for injection of the first contrast medium, with the first injection device 58 comprising a controller for carrying out a patient-specific injection protocol. The computed tomography system 31 also has a second injection device 60 for injection of the second contrast medium, with the second injection device 60 comprising a controller for carrying out a patient-specific injection protocol. The projection data acquisition unit 33 has a direct conversion X-ray detector in the detector device 29 or means for changing the X-ray spectrum or for adjusting the first energy range and the second energy range, for example via filtering or kV switching.

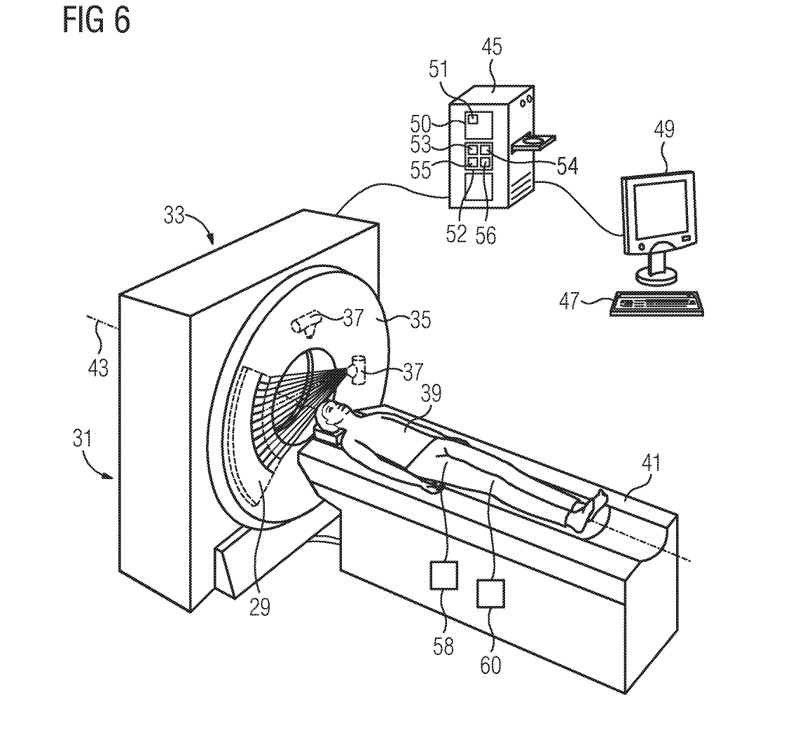

FIG. 6 shows an example implementation of an embodiment of the inventive computed tomography system 31 according to a second embodiment for carrying out embodiments of the inventive method. The projection data acquisition unit 33 has two X-ray sources 37 and two detector devices 29 (second detector device not shown for the sake of clarity). The computed tomography system 31 is a dual-source CT.

Although the invention has been illustrated in detail by the preferred example embodiment, it is not limited by the disclosed examples and a person skilled in the art can derive other variations herefrom without departing from the scope of the invention.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for imaging an examination region of an object to be examined with a computed tomography system, the examination region including a first contrast medium and a second contrast medium, different from the first contrast medium, the method comprising:
acquiring first projection scan data with a first energy range and second projection scan data, different from the first projection scan data, with a second energy range, the second energy range being different from the first energy range in the examination region; and
generating a first image based upon the first projection scan data acquired and the second projection scan data acquired, the first image including
only isolated first information of the first contrast medium,
only isolated second information of the second contrast medium, or
isolated first information of the first contrast medium and isolated second information of the second contrast medium.

2. The method of claim 1, wherein the first contrast medium has a therapeutically effective functionalization.

3. The method of claim 1, wherein the second contrast medium is indicative of a blood flow property.

4. The method of claim 1, wherein the first image includes an item of local information about a local distribution of at least one of the first contrast medium and second contrast medium.

5. The method of claim 1, wherein the first image includes an item of local information about a quantity of at least one of the first contrast medium and the second contrast medium.

6. The method of claim 1, wherein the first image includes an item of local information about a quantity or distribution of the first contrast medium having a therapeutically effective functionalization.

7. The method of claim 6, wherein the therapeutically effective functionalization includes at least one of chemical and radioactive substances bound to the first contrast medium.

8. The method of claim 1, wherein the first image includes an item of local information based on the second contrast medium about a local blood circulation in a region.

9. The method of claim 1, wherein the first contrast medium includes a contrast substance bound to microspheres or nanoparticles.

10. The method of claim 1, wherein the first energy range includes a first X-ray spectrum and the second energy range includes a second X-ray spectrum.

11. The method of claim 1, wherein the first energy range includes a first detector energy range and the second energy range includes a second detector energy range.

12. The method of claim 1, further comprising a step of multi-material analysis.

13. An image generating device comprising:
an input interface to acquire projection scan data from an examination region of an object to be examined, obtained via a computed tomography system with aid of a scan, the projection scan data including first projection scan data with a first energy range and second projection scan data, different from the first projection scan data, with a second energy range, the second energy range being different from the first energy range in the examination region;
a reconstruction unit to reconstruct a first image based upon the first projection scan data acquired and the second projection scan data acquired, the first image including
only isolated first information of the first contrast medium,
only isolated second information of the second contrast medium, or
isolated first information of the first contrast medium and isolated second information of the second contrast medium; and
an image data interface to output the first image.

14. A computed tomography system, comprising:
a projection data acquisition unit, including an X-ray source and a detector device to acquire the projection scan data of the examination region of the object to be examined;
a controller to control the projection data acquisition unit; and
the image generating device of claim 13.

15. A non-transitory computer program product storing a computer program, directly loadable into a storage device of a controller of a computed tomography system, the computer program including program segments to carry out the method of claim 1 when the computer program is executed in the controller of the computed tomography system.

16. A non-transitory computer-readable medium storing program segments, readable and executable by an arithmetic unit, to carry out the method of claim 1 when the program segments are executed by the arithmetic unit.

17. The method of claim 2, wherein the second contrast medium is indicative of a blood flow property.

18. The method of claim 2, wherein the first image includes an item of local information about a local distribution of at least one of the first contrast medium and second contrast medium.

19. The method of claim 2, wherein the first image includes an item of local information about a quantity of at least one of the first contrast medium and the second contrast medium.

20. The method of claim 2, wherein the first image includes an item of local information about a quantity or distribution of the first contrast medium having a therapeutically effective functionalization.

21. The method of claim 20, wherein the therapeutically effective functionalization includes at least one of chemical and radioactive substances bound to the first contrast medium.

22. A non-transitory computer program product storing a computer program, directly loadable into a storage device of a controller of a computed tomography system, the computer program including program segments to carry out the method of claim 4 when the computer program is executed in the controller of the computed tomography system.

23. A non-transitory computer-readable medium storing program segments, readable and executable by an arithmetic unit, to carry out the method of claim 4 when the program segments are executed by the arithmetic unit.
